(19) United States
(12) Patent Application Publication
(10) Pub. No.: US 2017/0189910 A1
(43) Pub. Date: Jul. 6, 2017

(54) SEALING CONNECTION ASSEMBLY FOR A SAMPLING DEVICE

(71) Applicant: PROVTAGAREN AB, Hässleholm (SE)

(72) Inventors: Gunnar SKARPING, Hässleholm (SE); Marianne DALENE, Hässleholm (SE)

(21) Appl. No.: 15/314,197

(22) PCT Filed: May 28, 2015
(86) PCT No.: PCT/SE2015/050616

§ 371 Date: Nov. 28, 2016

(30) Foreign Application Priority Data
May 28, 2014 (SE) 1450647-1

(51) Int. Cl. B01L 3/00; G01N 1/20; F16L 37/133; A61M 39/10; G01N 1/22

(52) U.S. Cl. CPC B01L 3/563; A61M 39/1011; B01L 3/502; G01N 1/2205; F16L 37/133; G01N 1/20; B01L 2200/087; B01L 2200/0689; B01L 2200/026; B01L 2300/0681; F16L 2201/44

(57) ABSTRACT

A sealing connection assembly for fluid-tightly sealing of a first tubular part (1) to a second tubular part (2), wherein the sealing connection assembly comprises a first locking element (3) being adapted to at least partly enclose said first tubular part (1) and comprising at least one gripping element (5), a second locking element (4) being adapted to at least partly enclose said second tubular part (2). The sealing connection assembly is characterized in that said at least one gripping element (5) of said first locking element (3) is adapted to sealingly engage said second locking element (4) thereby locking said first tubular part (1) and said second tubular part (2) together, and in that said sealing connection assembly further comprises a resilient washer (6) arranged between said first locking element (3) and said second locking element (4), said resilient washer (6) being adapted to apply a force between said first locking element (3) and said second locking element (4) in an axial direction of the sealing connection assembly when locked together. To be published:

### FIELD OF THE INVENTION

The present invention relates to a sealing connection assembly for fluid-tightly sealing of a first tubular part to a second tubular part. The invention further relates to a fluid-tightly sealable sampling device for environmental and medical analysis of one or more substances in a fluid flow intended to pass through the sampling device.

### BACKGROUND ART

In many application areas it is important to facilitate a fluid-tight sealing between different parts as e.g. when connecting to pipes to each other, when connecting two hoses, when securing a lid to a container, etc. The quality of the sealing in the sense that it is reliably fluid-tight also for long periods of time, is especially important in laboratory environments or in medical environments, since leakage could lead to faulting measurements, damage to equipment or wrong dosage or contamination of drugs to a patient.

One example is sealing, with a lid or cap, of a test tube for storing a gas or liquid, e.g. when taking a sample for later testing. The test tube may be transported long distances before it is reopened. To be able to rely on the sample in the test tube, the sealing of the test tube have to be fluid-tight also after a long time having endured e.g. pressure changes during air transport and temperature changes. Commonly used seals are mostly threaded, wherein e.g. a lid is screwed onto a test tube. This may result in a fluid tight sealing when the sealing is closed, but after a while, especially if temperature and pressure changes gave occurred, the materials of the sealing will give in to some extent and the sealing will leak.

One special example is in the field of air sampling using filters. In that case it is important to know that any filter, through which sampled air is intended to pass, is fluid tightly sealed. It is further important to have fluid tight sealing when closing the sampler after a measurement is done, so that no additional air enters the sampler, possibly contaminating the sampler before it arrives to a lab for analysis.

It is very important that the sealing of the filter of a sampler is fluid-tight and secure for the measurement to be reliable. If leakage occurs during the measurement in such a way that the gas flow may circumvent the filter, the measurement will be inaccurate. Currently used samplers show some structural drawbacks. E.g. the filter is held in place by a filter holder and is in contact directly with an abutment portion of the filter holder. By rotating either the filter holder or the adsorption tube, or both, when assembling the sampler components, the filter may be sheared or broken due to the rotational forces induced, and leaks may occur. The filter may also unintentionally shift in position during the rotation of the sampler components creating large gaps around the filter, thereby making the measurement inaccurate.

A further problem is that when storing the assembled sampler, the pressure exerted on the filter may alter due to ageing of the filter. Thereby the sealing properties may be negatively affected.

Before and after a sampling session has been performed it is also important that the sampling device is protected from the outside environment with a view to avoiding contamination via diffusion of undesired substances into the sampler. Therefore, it is important to use sealing caps in the inlet and outlet ends, in particular in the inlet end of the sampler when the sampler not is in use, e.g. during transport. Otherwise, the measurements may be negatively affected and destroyed by the undesired diffusion into the sampler. Thus, as sampling in several environments may be very expensive and require highly accurate measurement results, it is of great importance that the sampler is fluid-tight against the outside environment when assembled, in particular during handling and transport before and after the measurements.

Another problem in connection with use of sampling devices for the measurement of air-borne compounds in a fluid flow is the risk that the sampling device is tampered or manipulated during handling thereof, i.e. during the period from when it is transported from the supplier or the analysis laboratory to the user, is subjected to the sampling step by the user, and then is transported from the user to the analysis laboratory. When the sampling device is sent from the supplier or the analysis laboratory to the user, the filter is located within the sampling device, fluid tightly secured between the adsorption device and the filter holder. During the sampling step and the subsequent transport of the sampling device to the analysis laboratory the filter must be located within the sampling device all the time, i.e. the adsorption device and the filter holder may not be separated from each other. However, it has turned out that sampling devices have been manipulated or tampered with during the transports or by the user before, during, and after the sampling step, either unintentionally or intentionally. E.g., it has happened that the adsorption device has been separated from the filter holder during the transport of the sampling device to and from the user or by the user at the sampling site. In such a case the filter becomes exposed to air-borne components from other sites than the sampling sites and also during indefinite time periods. This of course leads to false or inaccurate analysis results in the end. The reason for such a manipulation could be that it is made by mistake or with a view to intentionally provide a different analysis result than the correct one. It has also happened that the filter has been exchanged with another filter containing the intended analyte components, i.e. reaction products, in intentionally wrong concentrations or having other compounds bound thereto.

Another problem is that when the sampling device has been used once it is further used one or more times after the analysis step of the laboratory. E.g., when the adsorption device has been separated from the filter holder and the filter has been taken out for analysis, it has happened, unintentionally or intentionally, that a new filter has been introduced in the filter holder and that the adsorption device thereafter has been connected to the filter holder, thereby creating a sampling device for repeated use. When such a sampling device is sent to a user for sampling, the interior surfaces thereof normally are contaminated with different compounds from the previous sampling, and the analysis results finally obtained at the analysis laboratory will be false or inaccurate. Such a manipulation can be made by mistake, e.g. when the different parts of the sampling device appear to be unused, or, in the intentional case, with a view to saving money by reuse thereof. U.S. Pat. No. 5,601,711 discloses a filter device for separation of materials, wherein it comprises two or more inline tubular elements, one or more of which is a module that houses a filter medium. The elements may have complementary connection structures, e.g. an o-ring, compression connections, bayonet connections, snap connections, and the like.

US 2010/0010455 discloses a medical delivery system adapted to be locked axially and unlocked rotationally.

US 2009/0242470 discloses a filter closure system having a connecting end and a connecting head which have a bayonet connection with receiving slots or receiving projections and matching insertion projections.

Thus, there is a clear need to provide an improved fluid-tightly seal for fluid connections where leakage is a concern, e.g. in lab environments, medical environments etc, both when it comes to transporting fluid through pipes and when it comes to transporting fluids in test tubes or similar containers.

There is also a need to provide an improved fluid-tightly seal without risk of any leakage around the edges of a filter of a sampling device, leakage of a lid of a test tube or leakage in the connection between two hoses or pipes. Further, there is a need for an improved sampler with a view to avoiding contamination from its surroundings during handling and transport of the sampling device and for a sampler having caps that are not lost during measurement and that not may contaminate the sampling device by the surroundings.

Thus, there is also a need for a way to prevent manipulation and tampering of the sampling device during the transport from the supplier or sampling laboratory to the user, by the user in connection with the sampling step, and during the transport from the user to the analysis laboratory. There is also a need to prevent use of the sampling device more than once.

### SUMMARY OF THE INVENTION

An object of the present invention is to eliminate the above-mentioned problems and provide a sealing connection assembly for a test tube lid, for a tube connection, for a sampling device for environmental, laboratory and medical analysis, etc, wherein improved sampling with higher liability in a fluid flow for the analysis of one or more substances of interest is provided and wherein the risk of manipulation and reuse of the sampling device is reduced or eliminated.

According to the invention, this object is achieved by means of a sealing connection assembly for fluid-tightly sealing of a first tubular part to a second tubular part, wherein the sealing connection assembly comprises a first locking element being adapted to at least partly enclose said first tubular part and comprising at least one gripping element, a second locking element being adapted to at least partly enclose said second tubular part. The sealing connection assembly is characterized in that said at least one gripping element of said first locking element is adapted to sealingly engage said second locking element thereby locking said first locking part and said second locking part together, and in that said sealing connection assembly further comprises a resilient washer arranged between said first locking element and said second locking element, said washer being adapted to apply a force in an axial direction of the sealing connection assembly between said first locking element and said second locking element when locked together.

The resilient washer will provide a force between the first and second locking element so that the sealing connection assembly provides a sealed connection also if the materials in the sealing connection assembly are affected by temperature and pressure changes. The skilled person realizes that the resilient washer can be any kind of resilient washer to provide the force mentioned above as e.g. a spring washer, a spring, a resilient rubber structure etc. In a preferred embodiment the resilient washer is a spring washer. A spring washer is thin, has a well defined spring force and is very robust providing a high quality solution. The resilient washer is preferably annular and is in one embodiment adapted to providing a force in the axial direction of the sealing connection assembly, said axial direction being the centre axis of the resilient washer. The axial direction is the direction parallel to the extension of the tubular part, i.e. in the fluid flow direction, when the first tubular part and the second tubular parts allow a fluid to flow though them.

The sealing connection assembly according to the invention may be used for any connection between pipes, channels, for caps/lids etc. where a fluid tight sealing is required. The first locking element and the second locking element have to be made of a material of high structural strength to not be affected by the constant force applied by the washer.

According to a further aspect of the invention said first tubular part and said second tubular part comprises an engagement element, respectively, wherein each of said respective engagement means are adapted to be interconnected when said first locking element and said second locking element are locked together, thereby preventing rotational movement between said first tubular part and said second tubular part.

Avoiding rotational movement between the first tubular part and the second tubular part eliminates possibilities of wear through frictional forces after assembling the sealing connection assembly. If placing, e.g. a filter between the first and second tubular parts, the filter will not be subjected to frictional forces that may harm the filter and cause leakage and unexpected results if used in measurements.

According to a further aspect of the invention the first locking element is tubular and may be integral with said first tubular part. The first tubular part may e.g. be the end of a tube or pipe, but it may also be a closed tube, i.e. a cap or lid.

The second locking element may further be integral with said second tubular part so as to reduce the number of parts needed in the sealing connection assembly. The sealing connection assembly may have a first tubular part that is constituted by a cap and a second tubular part is constituted by a test tube.

The sealing connection assembly may further be adapted to receive a fluid flow through said first tubular part and said second tubular part when locked together. This may e.g. be the case when using the sealing connection assembly for connecting to pipes or hoses.

According to a further aspect of the present invention the sealing connection assembly further comprises a filter device arranged between said first tubular part and said second tubular part, said filter device being adapted to divide the first tubular part from said second tubular part, such that a gas flow passing said sealing connection assembly will pass said filter device.

According to a further aspect of the inventive sealing connection assembly the first locking element is adapted to sealingly engage said second locking element by means of a snap in connection adapted to grip said second locking element with integral annularly placed clutch members, thereby locking said first locking part and said second locking part together. The clutches may grip around the second locking element, the second locking element being e.g. a rigid washer placed around the second tubular part, a groove in the second tubular part etc. The snap-in connection may be used if the sealing connection assembly is intended not to be opened by the user, e.g. in case of a sample test tube, where the test tube is to be used only once.

The clutches may be more than two and are preferably flexible so that they can bend slightly when being pushed over the edge of the second locking member. When the clutched have been pushed over the edge of the second locking member, the clutches bend back and grip around the second locking member. The same effect could be achieved with only one annular clutch being bendable enough to snap over the second locking element.

According to a further aspect of the present invention, the sealing connection assembly further comprises additional sealing elements between said first tubular part and said second tubular part, as e.g. an O-ring or a gasket for sealing purposes.

A further embodiment of the present invention is a fluid-tightly sealable sampling device for analysis of one or more substances in a fluid flow intended to pass through the sampling device, comprising a sealing connection assembly as discussed above. The first tubular part is in this embodiment an adsorption device which is hollow and is adapted to be provided with one or more reagents for adsorption of and reaction with said one or more substances in the fluid flow and the second tubular part comprises a filter holder for holding said filter device.

### BRIEF DESCRIPTION OF THE DRAWINGS

FIG. 1 schematically shows in an exploded view the different components of a sealing connection assembly according to the present invention used for a fluid-tightly sealable sampling device.

FIG. 2 schematically shows the sealing connection assembly according to FIG. 1 as assembled.

FIG. 3 schematically shows in an exploded view the different components of a sealing connection assembly according to the present invention used for connecting two hoses.

FIG. 4 schematically shows the sealing connection assembly according to FIG. 3 as assembled.

### DETAILED DESCRIPTION OF SPECIFIC EMBODIMENTS

The expression “fluid flow direction” used throughout the application text is intended to mean the axial direction in relation to the cross-section of the sealing connection assembly.

The expression “fluid flow” used throughout the application text is intended to mean a flow of a gas or a liquid, which also may contain components in solid form, e.g. fluidized particles and aerosols. One example of a fluid is an air flow containing small particles having the substances to analyze bound to their surfaces. Another example of a fluid flow is a water flow containing the substances to analyze, e.g. a drinking water flow, and flows in connection with purification plants.

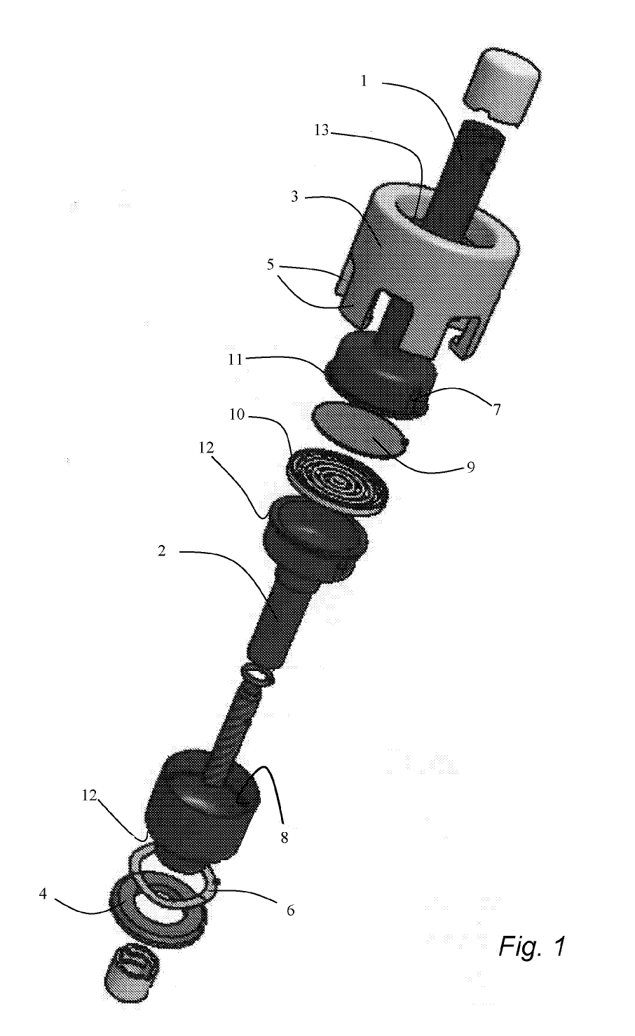

The present invention will now be disclosed more in detail with reference to FIG. 1, which shows a sealing connection assembly used for a sampling device. The first tubular part 1, the second tubular part 2, the filter device 9, and the filter holder 10 are pressed together in a fluid tight manner by the first locking element 3 gripping the second locking element 4 with clutches 5 in a snap in connection where the clutches 5 grip around the second locking element 4. The resilient washer 6 is compressed between the second locking element 4 and the second tubular part 2 so as to inflict a force in the axial direction of the sealing connection assembly and the sampling device. The annular first locking element 3 has an annular edge 13 working as a seat for the annular edge 11 of the first tubular part. The second tubular part has a corresponding annular edge 12 for receiving the resilient washer 6 and the second locking element 4. Looking carefully on the resilient washer 6 in the embodiment of FIG. 1, it can be noted that the resilient washer 6 is a spring washer 6.

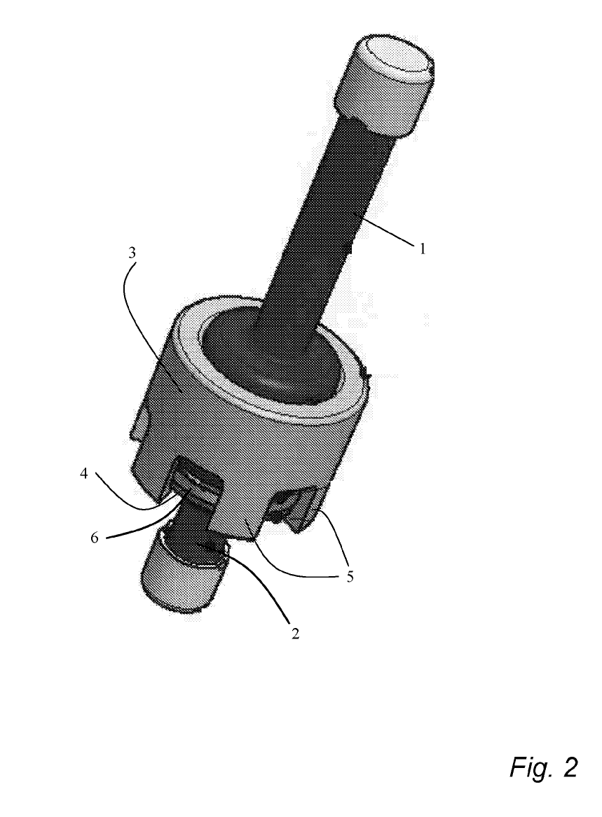

In FIG. 2 the sealing connection assembly of FIG. 1 is assembled. It is visible how the clutches 5, in a snap in connection, grip around the second locking element 4. The resilient washer 6 is compressed between the second locking element 4 and the second tubular part 2 applying a sealing force in the axial direction of the sealing connection assembly and the sampling device. The annular edge 13 of the first locking element 3 works as a seat for the annular edge 11 of the first tubular part. The annular edge 12 of the second tubular part receives the resilient washer 6 and the second locking element 4.

In FIG. 1 it is shown that the engagement element 7 on the first tubular part is adapted to engage the engagement element 8 on the second tubular part so as to eliminate any possibility of relative rotation around the axial direction between the first tubular part and the second tubular part. The engagement element 7 on the first tubular part is a projection that is adapted to be received in the engagement element 8 on the second tubular part. The engagement element 8 on the second tubular part is a slot adapted to receive the projection 7 of the first tubular part.

In FIG. 1 the second tubular part 2 is an adsorption device having a filter paper adjacent its inner wall. The sealing connection assembly is thus used as a sampling device in which a fluid flow may be drawn through by a suction pump (not shown) connected to the second tubular part. Substances and/or particles in the fluid flow may then be sampled either by being collected by the filter device 9 or being adsorbed to the filter in the adsorption device.

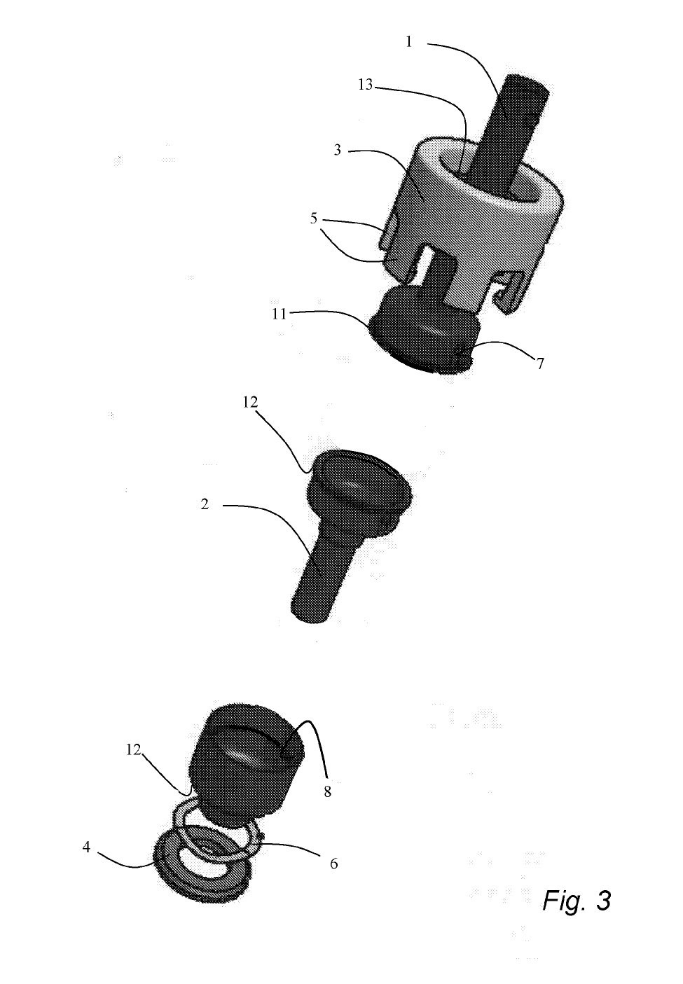

The present invention will now be disclosed more in detail with reference to FIG. 3, which shows a sealing connection assembly used for connecting two hoses 1, 2. The first tubular part 1 or first hose 1, the second tubular part 2 or hose 2 are pressed together in a fluid tight manner by the first locking element 3 gripping the second locking element 4 with clutches 5 in a snap in connection where the clutches 5 grip around the second locking element 4. The resilient washer 6 is compressed between the second locking element 4 and the second tubular part 2 so as to inflict a force in the axial direction of the sealing connection assembly and the sampling device. The annular first locking element 3 has an annular edge 13 working as a seat for the annular edge 11 of the first tubular part. The second hose 2 has a corresponding annular edge 12 for receiving the resilient washer 6 and the second locking element 4.

It is understood that the first tubular part 1 and the second tubular part 2 may be hose nipples for mounting the two respective hoses on.

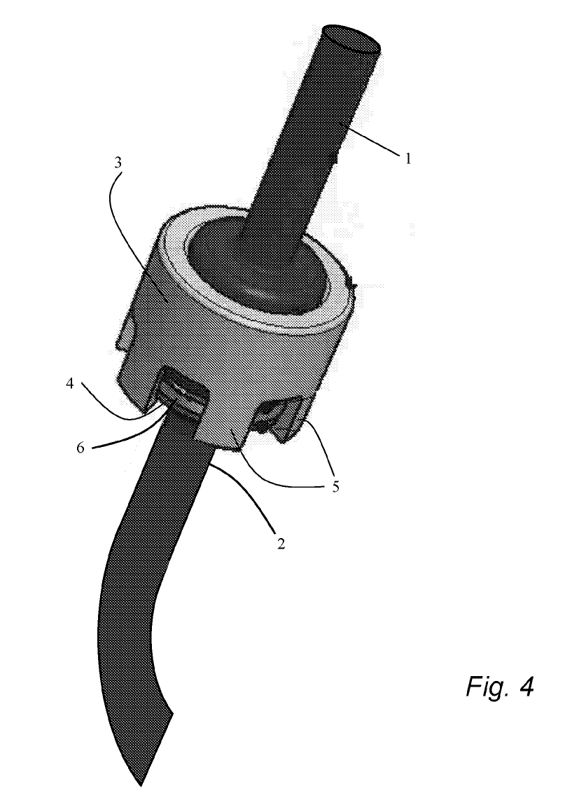

In FIG. 4 the sealing connection assembly of FIG. 3 is assembled. It is visible how the clutches 5, in a snap in connection, grip around the second locking element 4. The resilient washer 6 is compressed between the second locking element 4 and the second tubular part 2 applying a sealing force in the axial direction of the sealing connection assembly and the sampling device. The annular edge 13 of the first locking element 3 works as a seat for the annular edge 11 of the first tubular part. The annular edge 12 of the second tubular part receives the resilient washer 6 and the second locking element 4.

In FIG. 3 it is shown that the engagement element 7 on the first tubular part is adapted to engage the engagement element 8 on the second tubular part so as to eliminate any possibility of relative rotation around the axial direction between the first tubular part and the second tubular part. The engagement element 7 on the first tubular part is a projection that is adapted to be received in the engagement element 8 on the second tubular part. The engagement element 8 on the second tubular part 2 is a slot adapted to receive the projection 7 of the first tubular part 1. Looking carefully on the resilient washer 6 in the embodiment of FIG. 3, it can be noted that the resilient washer 6 is a spring washer 6.

It is understood that the embodiment of FIGS. 3 and 4 may easily be amended to instead accomplish a fluid-tightly sealed test tube. The first tubular part 1 is then a lid for the second tubular part 2, which is then the test tube. It is further understood that similar products in need of a fluid-tight sealing may be contemplated within the scope of the invention defined by the claims.

1. A sealing connection assembly for fluid-tightly sealing of a first tubular part (1) to a second tubular part (2), wherein the sealing connection assembly comprises
a first locking element (3) being adapted to at least partly enclose said first tubular part (1) and comprising at least one gripping element (5),
a second locking element (4) being adapted to at least partly enclose said second tubular part (2), wherein
said at least one gripping element (5) of said first locking element (3) is adapted to sealingly engage said second locking element (4) thereby locking said first tubular part (1) and said second tubular part (2) together, and
said sealing connection assembly further comprises a resilient washer (6) arranged between said first locking element (3) and said second locking element (4), said resilient washer (6) being adapted to apply a force between said first locking element (3) and said second locking element (4) in an axial direction of the sealing connection assembly when locked together.

2. The sealing connection assembly according to claim 1, wherein said first tubular part and said second tubular part comprises an engagement element (7, 8), respectively,
wherein each of said respective engagement elements (7, 8) are adapted to be interconnected when said first locking element (3) and said second locking element (4) are locked together, thereby preventing rotational movement between said first tubular part (1) and said second tubular part (2).

3. The sealing connection assembly according to claim 1,
wherein said first locking element (3) is tubular.

4. The sealing connection assembly according to claim 1, wherein said first locking element (3) is integral with said first tubular part (1).

5. The sealing connection assembly according to claim 1, wherein said second locking element (4) is integral with said second tubular part (2).

6. The sealing connection assembly according to claim 5, wherein said first tubular part (1) is constituted by a cap and said second tubular part (2) is constituted by a test tube.

7. The sealing connection assembly according to claim 1, wherein said sealing connection assembly is adapted to receive a fluid flow through said first tubular part (1) and said second tubular part (2) when locked together.

8. The sealing connection assembly according to claim 1, further comprising
a filter device (9) arranged between said first tubular part (1) and said second tubular part (2), said filter device (9) being adapted to divide the first tubular part (1) from said second tubular part (2), such that a gas flow passing said sealing connection assembly will pass said filter device (9).

9. The sealing connection assembly according to claim 1, wherein said first locking element (3) is adapted to sealingly engage said second locking element (4) by means of a snap in connection adapted to grip said second locking element with integral annularly placed clutch members (5), thereby locking said first locking part and said second locking (4) part together.

10. The sealing connection assembly according to claim 1, further comprising additional sealing elements between said first tubular part (1) and said second tubular part (2).

11. A fluid-tightly sealable sampling device for analysis of one or more substances in a fluid flow intended to pass through the sampling device, comprising a sealing connection assembly according to claim 1,
wherein said first tubular part (1) is an adsorption device which is hollow and is adapted to be provided with one or more reagents for adsorption of and reaction with said one or more substances in the fluid flow
and wherein said second tubular part (2) comprises a filter holder (10) for holding said filter device (9).
